(19) United States
(12) Patent Application Publication
(10) Pub. No.: US 2022/0110682 A1
(43) Pub. Date: Apr. 14, 2022

(54) Computer-Implemented Method, Computer-Assisted Processing Device and Non-Transitory Computer-Readable Medium for Computer-Assisted Planning of Surgical Path

(71) Applicant: National Central University, Taoyuan City (TW)

(72) Inventors: CHING SHIOW TSENG, Taoyuan City (TW); JHENG-RUEI YU, Taoyuan City (TW)

(73) Assignee: National Central University, Taoyuan City (TW)

(21) Appl. No.: 17/074,998

(22) Filed: Oct. 20, 2020

(30) Foreign Application Priority Data
Oct. 8, 2020 (TW) 109135020

(51) Int. Cl. A61B 34/10; A61B 34/00; G06T 7/00; G06T 15/83; G06T 19/00; G06T 19/20

(52) U.S. Cl. CPC A61B 34/10; A61B 34/25; G06T 7/0012; G06T 15/83; G06T 19/003; G06T 19/20; G06T 2219/2016; A61B 2034/105; A61B 2034/107; G06T 2207/10072; G06T 2207/30096; G06T 2207/20182; A61B 2034/104

(57) ABSTRACT

The present invention relates to a computer-implemented method for a computer-assisted planning of a surgical path. The method includes accessing a data set of a computer-readable three-dimensional visualization medical representation including at least one three-dimensional medical model; loading the data set into and presenting the data set in a three-dimensional surgical path simulator; inserting a virtual three-dimensional object representing for the surgical path and features thereof into the data set; and adjusting a relative position of the virtual three-dimensional object with respect to the at least one three-dimensional medical model.

### CROSS-REFERENCE TO RELATED APPLICATION

This application claims priority benefit to Taiwan Invention Patent Application Serial No. 109135020, filed on Oct. 8, 2020, in Taiwan Intellectual Property Office, the entire disclosures of which are incorporated by reference herein.

### FIELD

The present invention relates to a computer-implemented method, computer-assisted processing device and non-transitory computer-readable medium for computer-assisted planning of surgical path, in particular to a computer-implemented method, computer-assisted processing device and computer program product for computer-assisted planning of surgical path that are incorporable with a computer-assisted surgery or a robotic-assisted surgery.

### BACKGROUND

In computer-assisted surgery technology, the computer-assisted planning of surgical path is implemented during periods of time before and after the patient is anesthetized before the surgical operation, the vicinity of the target lesion is scanned by the C-arm X-ray fluoroscopy machine or the MRI machine, and the scanned images are displayed on the surgical navigation software. The conventional surgical navigation software provides four standard windows, three of which are section windows to present the images of the target lesion section orthogonal to the sagittal, coronal and axial reference sections, and the doctor can manually set the positions and the angles of these three section windows. The window other than the three windows usually presents a 3D lesion model.

Each of the three section windows is configured with a sliding button on the edge thereof, and when the doctor drags the sliding button to issue an instruction, the software immediately drives the C-arm to move to the specified position and angle to create three new reference sections, to scan the computerized tomography (CT) images of the target lesion based on the new reference sections, and transmit the images back to the section windows for display in response to the doctor's operation, so that the doctor can define the reference section at any time by manually adjusting and changing the position and angle of the individual reference section. By operating the section windows, the doctor can view the basic physiological structures of the target lesion and the part near the target lesion to find the appropriate entry point for surgery and the surgical path between the entry point to the lesion (target point). The experienced doctor usually operates the section windows to control the C-arm to continuously rotate around the lesion; from the perspective of the doctor, the above operation is equivalent to the operation of the doctor holding a camera to continuously shoot around the target lesion, thereby quickly finding the entry point and the surgical path.

The above-mentioned planning of surgical path seems easy, but the CT images displayed on the three section windows are only two-dimensional planar images and lack information such as the distributions of arteries, veins, nerve plexus and nerve lines and so on. Therefore, during the actual operation, the doctor may accidentally injure the arteries or nerves to cause blood loss and nerve damage during the operation, or even severe sequelae or even death of the patient. Moreover, because the patient is already anesthetized, it may not be possible for the doctor to think carefully under time pressure. Furthermore, a lot of X-ray images must be taken during the above-mentioned operation process, so the patient is exposed to a lot of radiation and is very unsafe.

Hence, there is a need to solve the above deficiencies/issues.

### SUMMARY

In order to well solve the conventional technical issues, the present invention proposes a method for computer-assisted planning of surgical path, a computer-assisted processing device and a computer program product, which is able to reconstruct a three-dimensional digital medical model based on a series of two-dimensional medical slide images, to clearly present the spatial distribution of blood vessels, nerves, and other important human tissues around the target lesion, and accurately present the location of the lesion.

Accordingly, the present invention provides a computer-implemented method for a computer-assisted planning of a surgical path. The method includes accessing a data set of a computer-readable three-dimensional visualization medical representation including at least one three-dimensional medical model; loading the data set into and presenting the data set in a three-dimensional surgical path simulator; inserting a virtual three-dimensional object representing for the surgical path and features thereof into the data set; and adjusting a relative position of the virtual three-dimensional object with respect to the at least one three-dimensional medical model.

The present invention further provides a non-transitory computer-readable medium storing a program causing a computer to execute a computer-implemented method for a computer-assisted planning of a surgical path. The method includes in response to a user's operation, accessing a data set of a computer-readable three-dimensional visualization medical representation including at least one three-dimensional medical model; loading the data set into and presenting the data set in a three-dimensional surgical path simulator; inserting a virtual three-dimensional object representing for the surgical path and features thereof into the data set; and adjusting a relative position of the virtual three-dimensional object with respect to the at least one three-dimensional medical model.

The present invention further provides a computerized processing device for a computer-assisted planning of a surgical path. The device includes a processing unit configured to load and execute a three-dimensional surgical path simulator, in which the three-dimensional surgical path simulator accesses a data set of a computer-readable three-dimensional visualization medical representation including at least one three-dimensional medical model in response to a user's operation; a display unit configured to present the three-dimensional surgical path simulator and the data set; and an input unit configured to provide for a user to operate to insert a virtual three-dimensional object representing for the surgical path and features thereof into the data set and adjust a relative position of the virtual three-dimensional object with respect to the at least one three-dimensional medical model.

The above content described in the summary is intended to provide a simplified summary for the presently disclosed invention, so that readers are able to have an initial and basic understanding to the presently disclosed invention. The above content is not aimed to reveal or disclose a comprehensive and detailed description for the present invention, and is never intended to indicate essential elements in various embodiments in the present invention, or define the scope or coverage in the present invention.

### DESCRIPTION OF THE DRAWINGS

A more complete appreciation of the invention and many of the attendant advantages thereof are readily obtained as the same become better understood by reference to the following detailed description when considered in connection with the accompanying drawing, wherein:

FIG. 1 is a schematic diagram illustrating a system architecture of the computer-assisted processing system for computer-assisted planning of surgical path, according to the present invention;

FIG. 2 is a schematic diagram illustrating the medical data set server 200 and computer-assisted processing device for the computer-assisted planning of surgical path included in the computer-assisted processing system according to the present invention;

FIG. 3 is a schematic diagram illustrating the three-dimensional brain vascular medical model accessible for the three-dimensional surgical path simulator according to the present invention;

FIG. 4 is a schematic diagram illustrating the integrated medical model for target lesion accessible for the three-dimensional surgical path simulator according to the present invention;

FIG. 5 is a schematic diagram illustrating the graphical user interface included in the three-dimensional surgical path simulator according to the present invention;

FIG. 6 is a schematic diagram illustrating the operation panel component included in the three-dimensional surgical path simulator according to the present invention;

FIGS. 7 to 12 are schematic diagrams illustrating the actual operation process of preoperative surgical path planning of cerebral hemangioma surgery applying the first embodiment of three-dimensional surgical path simulator according to the present invention;

FIGS. 13 to 16 are schematic diagrams illustrating the actual operation process of preoperative surgical path planning of the aneurysm surgery applying the second embodiment of the three-dimensional surgical path simulator according to the present invention;

FIG. 17 is a schematic diagram illustrating the third embodiment of three-dimensional surgical path simulator according to the present invention;

FIG. 18 is a flow chart illustrating the computer-implemented method for a computer-assisted planning of a surgical path according to the present invention;

FIG. 19 is a schematic diagram illustrating the scenario applying the surgical path planned by the present invention in an augmented reality surgical navigation system, and a schematic diagram of doctor's AR view in which the surgical path planned by the present invention, is overlapped with the reality; and

FIG. 20 is a schematic diagram illustrating that the surgical path planned by the present invention is projected and overlapped with the reality through AR surgical navigation system.

### DETAILED DESCRIPTION

The present disclosure will be described with respect to particular embodiments and with reference to certain drawings, but the disclosure is not limited thereto but is only limited by the claims. The drawings described are only schematic and are non-limiting. In the drawings, the size of some of the elements may be exaggerated and not drawn on scale for illustrative purposes. The dimensions and the relative dimensions do not necessarily correspond to actual reductions to practice.

It is to be noticed that the term “including”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device including means A and B” should not be limited to devices consisting only of components A and B.

The disclosure will now be described by a detailed description of several embodiments. It is clear that other embodiments can be configured according to the knowledge of persons skilled in the art without departing from the true technical teaching of the present disclosure, the claimed disclosure being limited only by the terms of the appended claims.

The data set of the computer-readable three-dimensional visualization medical representation described in the present invention is abbreviated as the three-dimensional medical data set or medical data set, and includes the generalized set of three-dimensional medical models, graphics or layers related to structural and spatial distributions of bones, skins, lesion details, body contours, tissues, organs, arteries and veins around the specific target lesion. The three-dimensional medical model included in each medical data set can be superimposed after calibration and alignment.

These three-dimensional medical models are pre-constructed independently by implementing a modelling algorithm in data pre-processing. The modelling algorithm uses various medical perspective slide images as the original medical image data, and the medical perspective slide images includes, for example but not limited to: X-ray images, computerized tomography (CT) images, computerized tomography angiography (CTA) images, digital subtraction angiography (DSA) images, maximum intensity projection (MIP) images, magnetic resonance imaging (MRI) images or magnetic resonance angiography (MRA) images, functional anisotropy (FA) images, diffusion tensor imaging (DTI) images or diffusion-weighted imaging (DWI) images; and these images are constructed or reconstructed as at least one three-dimensional medical model through a series of processes including noise removal process, image pre-processing, anatomy based feature recognition, anatomy based feature enhancement, alignment process, stitching process, interpolation or extrapolation, so as to form the three-dimensional medical data set. The three-dimensional medical data set is pre-stored in the medical data set server 200 in a remote end or a non-volatile memory on the local device for further access.

After the modeling of the three-dimensional medical model is completed and the three-dimensional medical model is then calibrated and aligned, the three-dimensional medical model alignment and merging algorithm can be further implemented to selectively position and align the medical models, and then merge or superimpose the processed medical models into a single integrated medical model 270 for a target lesion and for further access.

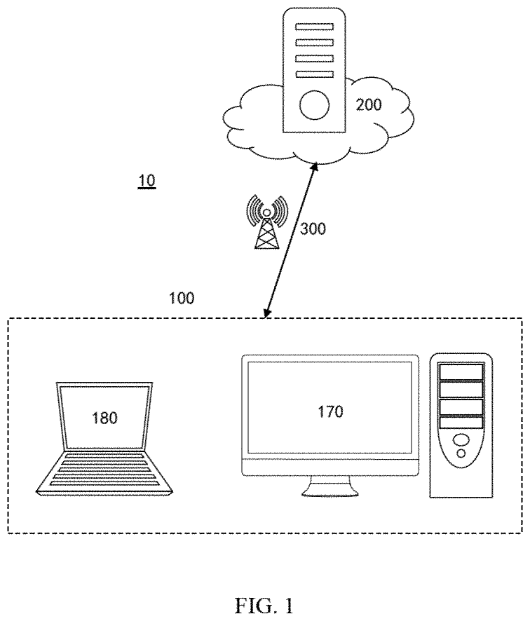

FIG. 1 is a schematic diagram illustrating a system architecture of the computer-assisted processing system for computer-assisted planning of surgical path, according to the present invention. The computer-assisted processing system 10 includes multiple hardware devices and computer program products. The hardware devices belonging to the physical devices can include, for example but not limited to, the multiple computer-assisted processing devices 100 according to the present invention located at a local end and a medical data set server 200 in a remote end. The computer-assisted processing devices 100 can be, for example but not limited to: desktop computers 170 or notebook computers 180. The local user device such as the desktop computer 170 or the notebook computer 180 can be selectively linked to the medical data set server 200 in a remote end through internet 300, to communicate with the medical data set server 200 in a remote end to access data in bidirectional. The internet 300 can be selected from one of local area network (LAN), wide area network (WAN), GSM network, 4G network, 5G network, 6G network, Wi-Fi network, Bluetooth network and a combination thereof.

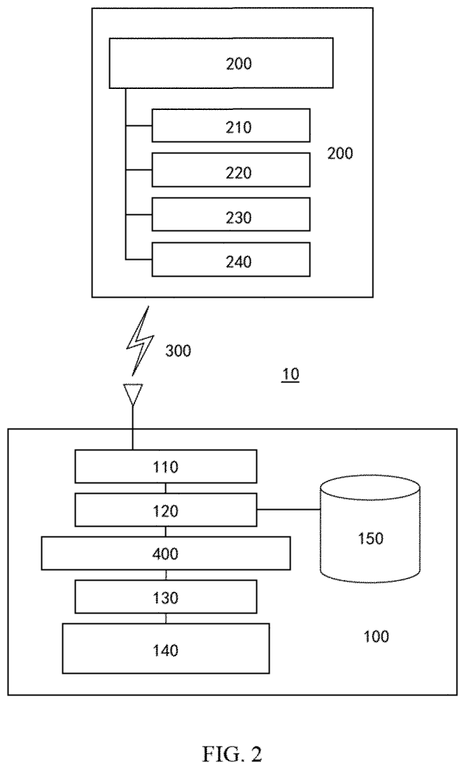

FIG. 2 is a schematic diagram illustrating the medical data set server 200 and computer-assisted processing device for the computer-assisted planning of surgical path included in the computer-assisted processing system according to the present invention. The computer-assisted processing device 100 according to the present invention includes hardware units such as a communication module 110, a processing unit 120, and a three-dimensional surgical path simulator 400, an input unit 130, a display unit 140, and a non-volatile memory 150 which are electrically connected to each other, and software components installed in the three-dimensional surgical path simulator 400 and including, for example, graphical user interface, viewable zone component for medical model, operation panel component, and three-dimensional medical graphics processing program module. Preferably, the three-dimensional surgical path simulator 400 is installed in the computer-assisted processing device 100 and can be loaded and executed by the processing unit 120.

The medical data set server 200 in a remote end stores the three-dimensional medical data set including, for example but not limited to: a skin medical model 210, a vascular medical model 220, a neural medical model 230, and a skeleton medical model 240 and so on. According to different surgical requirements, the medical data set server 200 stores more various medical models, the three-dimensional surgical path simulator 400 is linked to the medical data set server 200 through the communication module 110, to access the skin medical model 210, the vascular medical model 220, the neural medical model 230 or the skeleton medical model 240.

The present invention can also be implemented on the computer-assisted processing device 100 in standalone architecture, and the three-dimensional medical data set including the skin medical model 210, the vascular medical model 220, the neural medical model 230, the skeleton medical model 240, the three-dimensional cerebrovascular medical model 250, the three-dimensional skull medical model 260 and the integrated medical model 270 for a target lesion is stored in the non-volatile memory 150 of the computer-assisted processing device 100. The processing unit 120 of the computer-assisted processing device 100 can access the non-volatile memory 150 to acquire the three-dimensional medical data set, and load the three-dimensional medical data set into the three-dimensional surgical path simulator 400.

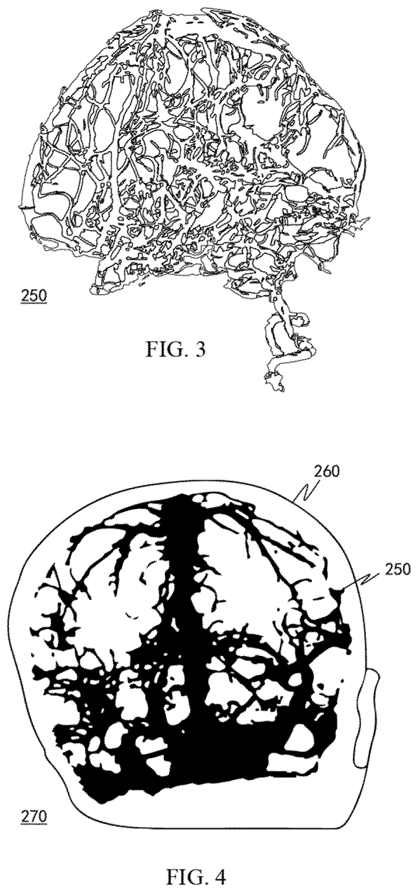

FIG. 3 is a schematic diagram illustrating the three-dimensional brain vascular medical model accessible for the three-dimensional surgical path simulator according to the present invention, and FIG. 4 is a schematic diagram illustrating the integrated medical model for target lesion accessible for the three-dimensional surgical path simulator according to the present invention. In this embodiment, the craniotomy is taken as an example to illustrate the implementation according to the present invention, and preferably, the three-dimensional medical data set includes, for example but not limited to, three-dimensional cerebrovascular medical model 250 shown in FIG. 3 and the integrated medical model 270 for a target lesion shown in FIG. 4. The integrated medical model 270 for a target lesion is formed by pre-implementing the three-dimensional medical model aligning and merging algorithm to merge the three-dimensional cerebrovascular medical model 250 and the three-dimensional skull medical model 260. Preferably, the three-dimensional medical data set includes, for example but not limited to: the sulcus medical model, the brain neural medical model, the brain wrinkle medical model, the integrated brain medical model, the aneurysm medical model, the cerebral hemorrhage medical model or the cerebral edema medical model.

For example, the three-dimensional cerebrovascular medical model 250 is preferably the three-dimensional digital medical model providing information about the distribution of the patient's cerebral arteries and veins, and the three-dimensional skull medical model 260 is preferably the three-dimensional digital medical model providing information about the structure of the patient's skull. The integrated medical model 270 for a target lesion is a medical model constructed by superimposing the three-dimensional cerebrovascular medical model 250 and the three-dimensional skull medical model 260. The three-dimensional cerebrovascular medical model 250, the three-dimensional skull medical model 260 and the integrated medical model 270 for a target lesion are pre-constructed and selectively stored on the medical data set server 200 in a remote end for further access.

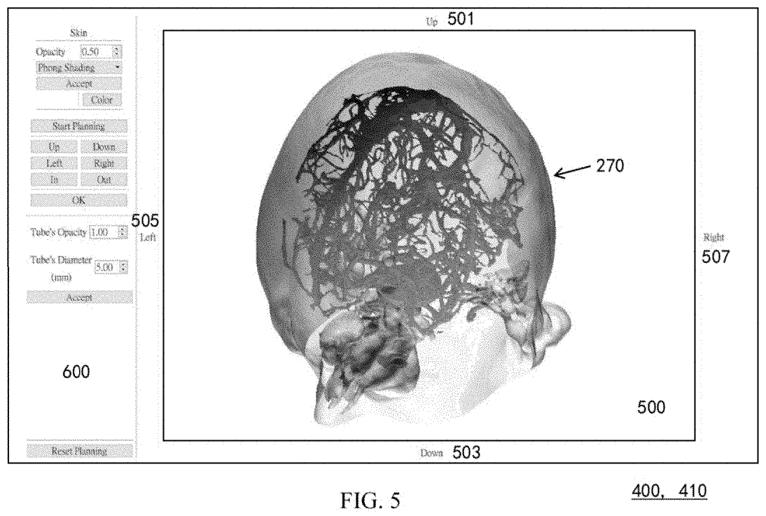

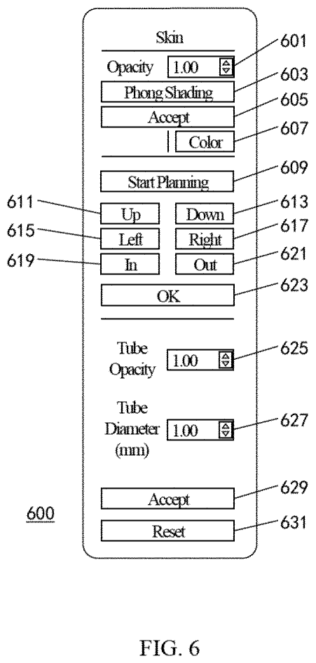

FIG. 5 is a schematic diagram illustrating the graphical user interface included in the three-dimensional surgical path simulator according to the present invention, and FIG. 6 is a schematic diagram illustrating the operation panel component included in the three-dimensional surgical path simulator according to the present invention. The three-dimensional surgical path simulator 400 according to the present invention includes a graphical user interface 410 for the surgeon to view, operate and edit. The graphical user interface 410 is configured with a viewable zone component 500 for medical model, and an operation panel component 600 configured closely adjacent to the viewable zone component 500.

The three-dimensional surgical path simulator according to the present invention is belonged to the computer program product of application layer or software platform, and includes several program modules including software components, such as but not limited to, a graphical user interface 410, a viewable zone component 500 for medical model, an operation panel component 600 and a three-dimensional medical graphics processing program module. Preferably, the medical data set stored on medical data set server 200 in a remote end is loaded into the three-dimensional surgical path simulator through a series of graphical user interfaces, so as to present the target lesion medical model, the vascular medical model, or the neural medical model for doctors to view, edit, modify, and plan a safe surgical path. The surgical path is a path from a surgical entry point (incision) to the target point (lesion), and is usually a straight line.

The operation panel component is embedded in the user interface of the three-dimensional surgical path simulator, and preferably, the operation panel component is configured on the left or right side of the three-dimensional surgical path simulator. The operation panel component has a variety of functional buttons for a user to issue an operation instruction to operate the three-dimensional surgical path simulator, so as to operate the target lesion medical model, the vascular medical model or the neural medical model to zoom in/zoom out, magnify/shrink, resize, translate, rotate, adjust viewing angle, change degree of opacity, edit, modify, disable layer or enable layer. The three-dimensional medical graphics processing program module is configured to process the graphics computation process required by the target lesion medical model, the vascular medical model, or the neural medical model loaded into the three-dimensional surgical path simulator.

In this embodiment, the integrated medical model 270 for a target lesion is loaded into the viewable zone component 500 for medical model and presented as the visualization three-dimensional medical model for doctors to view. The integrated medical model 270 for a target lesion includes the target lesion medical model and arterial medical model for the target lesion, the doctor can insert the virtual three-dimensional object representing for the surgical instrument into the viewable zone component 500 for medical model by inputting the operation instruction, and then move the virtual three-dimensional object in the viewable zone component for medical model according to the presentation of the three-dimensional medical model. During the above-mentioned process, the doctor just needs to visually observe and adjust the positional relationship of the virtual three-dimensional object with respect to the three-dimensional medical model, and intuitively determine whether the virtual three-dimensional object may cut the three-dimensional medical model, so as to find one or more feasible surgical path.

The doctor first loads the target lesion medical model, the vascular medical model or the neural medical model into the viewable zone component 500 for medical model, the viewable zone component 500 has orientation indicators marked on the four edges thereof, for example, the orientation indicators include an up indicator 501, a down indicator 503, a left indicator 505, and a right indicator 507. The three-dimensional surgical path simulator (computer program product) according to the present invention allows the doctor to input an operation instruction through the input unit such as a mouse or a keyboard. After the medical model is loaded into the viewable zone component 500 for medical model, the doctor can input the operation instruction through the mouse or the keyboard, to control the presentation of the medical model, for example but not limited to, the doctor can long press the left button of the mouse and drag the medical model to rotate in a specific direction or angle, or long press the right button of the mouse and drag the medical model to translate in a specific direction, or press and hold the ctrl button of the keyboard and upwardly rotate the wheel of the mouse to zoom in the medical model, or press and hold the ctrl button of the keyboard and downwardly rotate the wheel of the mouse to zoom out the medical model, as shown in FIG. 5.

The three-dimensional surgical path simulator according to the present invention further provides the doctor to input the operation instruction through the operation panel component 600. The operation panel component 600 includes a row of control buttons or fields representing the operation instructions. The buttons and fields of issuing operation instructions for the medical model can include, for example but not limited to: the field 601 of setting a degree of opacity for presentation of the medical model, the Phong shading button 603 of enabling a Phong shading for presentation of the medical model, the Accept button 605 of accepting the setting for the presentation of the medical model, and the Color button 607 of enabling three-dimensional rendering for the presentation of the medical model.

The buttons and fields of issuing operation instruction for planning of the surgical path, the Start Planning button 609 of the operation to start the planning of the surgical path and inserting the virtual three-dimensional object into the viewable zone component 500 for medical model, the Up button 611 of the operation to upwardly move the virtual three-dimensional object, the Down button 613 of operation to downwardly move the virtual three-dimensional object, the Left button 615 of operation to leftwardly move the virtual three-dimensional object, the Right button 617 of the operation to rightwardly move the virtual three-dimensional object, the In button 619 of the operation to zoom in, the Out button 621 of operation to zoom out, the OK button 623 of operation to accept the setting for the object, the Tube Opacity field 625 of operation to set the degree of opacity of the virtual three-dimensional object, the Tube Diameter field 627 of operation to set the tube diameter of the virtual three-dimensional object, the Accept button 629 of operation to accept the setting for the object, the Reset button 631 of operation to reset, as shown in FIG. 6.

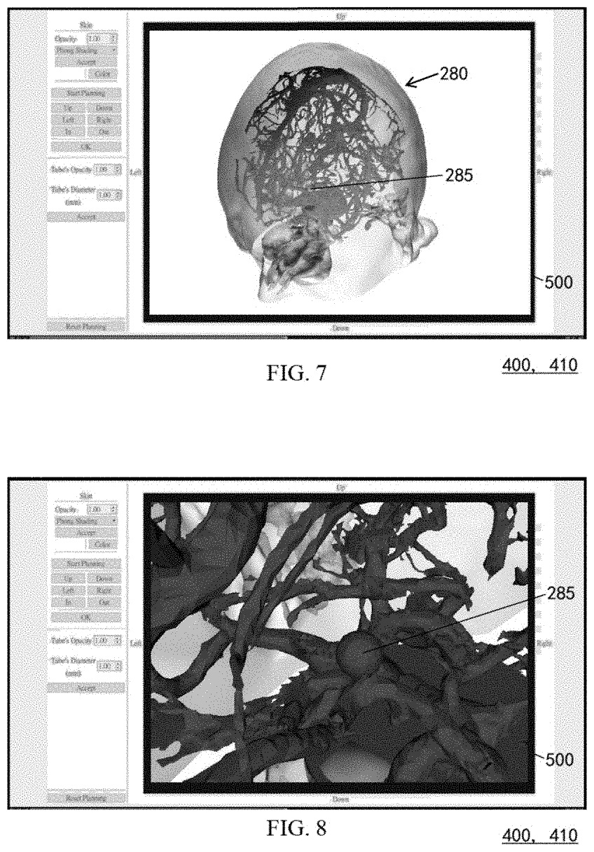

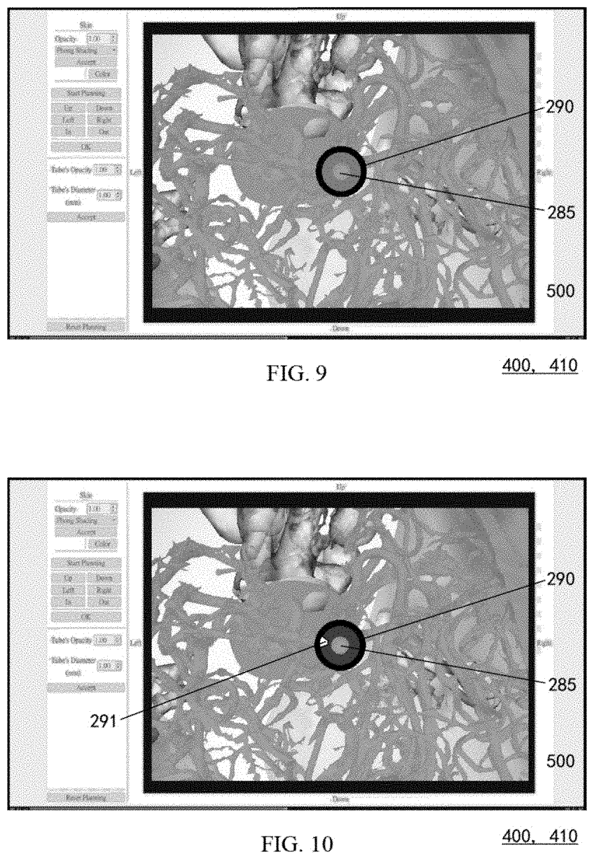

FIGS. 7 to 12 are schematic diagrams illustrating the actual operation process of preoperative surgical path planning of cerebral hemangioma surgery applying the first embodiment of three-dimensional surgical path simulator according to the present invention. In this embodiment, the graphical user interface 410 of the three-dimensional surgical path simulator 400 according to the present invention includes single viewable zone component 500 (or the three-dimensional medical model display window) for medical model, and the aneurysm surgery is taken as an example to illustrate the implementation process of the three-dimensional surgical path simulator according to the present invention; however, the implementation according to the present invention is not limited to aneurysm surgery, and the present invention can be applied to all types of surgery requiring pre-planning of the surgical path. Before the doctor performs the aneurysm surgery, the doctor can use the three-dimensional surgical path simulator according to the present invention to implement the planning of the surgical path.

In this embodiment, the doctor first superimposes and integrates the patient's three-dimensional cerebrovascular medical model 250, the three-dimensional skull medical model 260, and the three-dimensional brain lesion medical model by implementing the three-dimensional medical model alignment and merging algorithm to form the integrated medical model of the integrated brain medical model 280, and then load the integrated brain medical model 280 into the viewable zone component for medical model 500 of the graphical user interface 410 in the three-dimensional surgical path simulator according to the present invention. The correct position of the lesion, that is, the cerebral hemangioma 285, is clearly indicated in the patient integrated brain medical model 280 presented in the viewable zone component for medical model 500, as shown in FIG. 7.

Next, the doctor can input the operation instruction to the three-dimensional surgical path simulator through the mouse or the keyboard or through one of the plurality of buttons on the operation panel component 600 or directly using mouse to operate the viewable zone component for medical model 500 with dragging operation, to rotate the patient's integrated brain medical model 280 to any direction and angle, so as to view the actual spatial distribution of blood vessels, nerve plexuses, tissues or organs around the lesion from different angle or position. The doctor can view the lesion and its surroundings from, for example but not limited to, the front direction, the front left direction or the front right direction, and adjust the degree of opacity and enable Phong shading to observe the conditions of the lesions and surroundings in detail.

When the doctor watches the lesion and the surrounding conditions from a certain view angle, if the doctor finds a feasible surgical path existed along the doctor's visual line of sight to the lesion and there are relatively few and sparse distribution of blood vessels, nerve plexuses, internal tissues and organs around the found surgical path, for example, the physician first directly presses the ctrl key of the keyboard and the scroll wheel of the mouse to zoom in the integrated brain medical model 280 in the viewable zone component for medical model 500 by using the cerebral hemangioma 285 as the central point, as shown in FIG. 8.

after the doctor operates the button 609 to enable planning on the operation panel component 600 or directly selects the lesion (that is, the aneurysm 285) by clicking the left button of the mouse in the viewable zone component for medical model 500, the system inserts the tubular virtual three-dimensional object 290 into the patient's integrated brain medical model 280 in the viewable zone component 500 for medical model, wherein the tubular virtual three-dimensional object 290 represents for the surgical path using the aneurysm 285 as a starting point and positioned along the doctor's visual line of sight. The system presets the virtual three-dimensional object 290 is substantively parallel to the doctor's visual line of sight, so the virtual three-dimensional object 290 looks like a circle when just being inserted into the integrated brain medical model 280, as shown in FIG. 9.

In this embodiment, the system presets the diameter of the virtual three-dimensional object 290 as 1 cm, and the diameter of 1 cm is a slightly larger diameter, so the virtual three-dimensional object 290 looks like to enclose intracranial aneurysm 285, as shown in FIG. 9; however, the doctor can change the diameter of the virtual three-dimensional object 290 by inputting the appropriate value of tube diameter in the object diameter setting field 627 according to the planning of the operation, to change the diameter of the virtual three-dimensional object 290.

The doctor can to fine tune the direction of the virtual three-dimensional object 290 about the aneurysm 285 by pressing the Up button 611 of operation to rotate the operation panel component 600 upwardly, the Down button 613 of operation to rotate the operation panel component 600 downwardly, the Left button 615 of operation to rotate the operation panel component 600 leftwardly, or the Right button 617 of operation to rotate the operation panel component 600 rightwardly in the operation panel component 600. For example, the doctor can press the Right button 617 to rightwardly rotate the position of virtual three-dimensional object 290 slightly, or the Down button 613 to downwardly rotate the position of the virtual three-dimensional object 290 slightly.

At any time point during operation, when the three-dimensional medical graphics processing engine program module included in the three-dimensional surgical path simulator determines that an intersect condition occurs between tissues, blood vessels and nerve plexus of the integrated brain medical model 280 and the tube's diameter of the virtual three-dimensional object 290 based on spatial geometric calculation, it means that the surgical instrument is likely to cut or injure blood vessels or nerve plexus when the surgery is performed along this surgical path; at this time, the three-dimensional surgical path simulator immediately highlight the zone, where the virtual three-dimensional object 290 and the medical model are intersected with each other, as an anomalistic block 291 by a hollow pattern, an inverted color or a special color or flashing manner, so as to give immediate feedback to the doctor. The doctor can evaluate whether this surgical path is feasible, or operate one of the buttons on the operation panel component 600 to re-adjust the direction, the position or tube's diameter of the virtual three-dimensional object 290, or click the reset planning button 631 to start a new planning of surgical path.

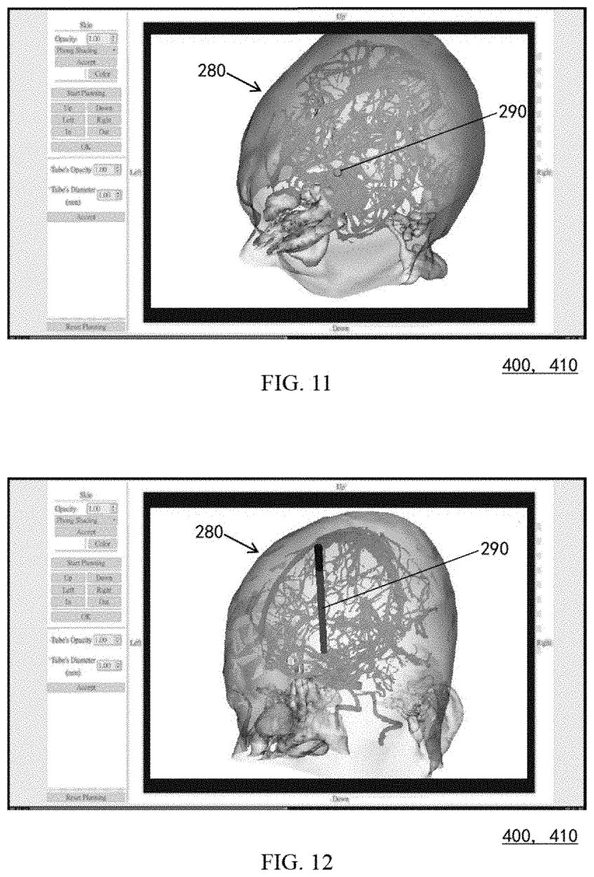

After the doctor initially finds out the surgical path, it indicates that the position, orientation and tube diameter of the virtual three-dimensional object 290 and other conditions are completely set in initial. Next, the doctor can continue to use the mouse or the keyboard to issue operation instruction to the viewable zone component for medical model 500 of the three-dimensional surgical path simulator, to zoom out the integrated brain medical model 280 together with the virtual three-dimensional object 290, as shown in FIG. 11; the virtual three-dimensional object 290 is still parallel to the doctor's sight, so the virtual three-dimensional object 290 looks like a circle. Next, the doctor drags the mouse to rotate the integrated brain medical model 280 together with the virtual three-dimensional object 290, so as to observe the virtual three-dimensional object 290 (that is, the planned surgical path) from different direction, for example, the doctor views the preliminary plan surgical path from the front of the head (based on the direction of the ear), and the doctor can clearly see the condition of the surgical path passing through the brain tissues, blood vessels, nerve plexus from the three-dimensional perspective, as shown in FIG. 12.

After the doctor confirms the planning of the surgical path, the doctor clicks the Accept button 629 on the operation panel component 600 of the three-dimensional surgical path simulator, and the system then saves the planned surgical path and related data to the medical data set server 200 in remote or the non-volatile memory on the local device.

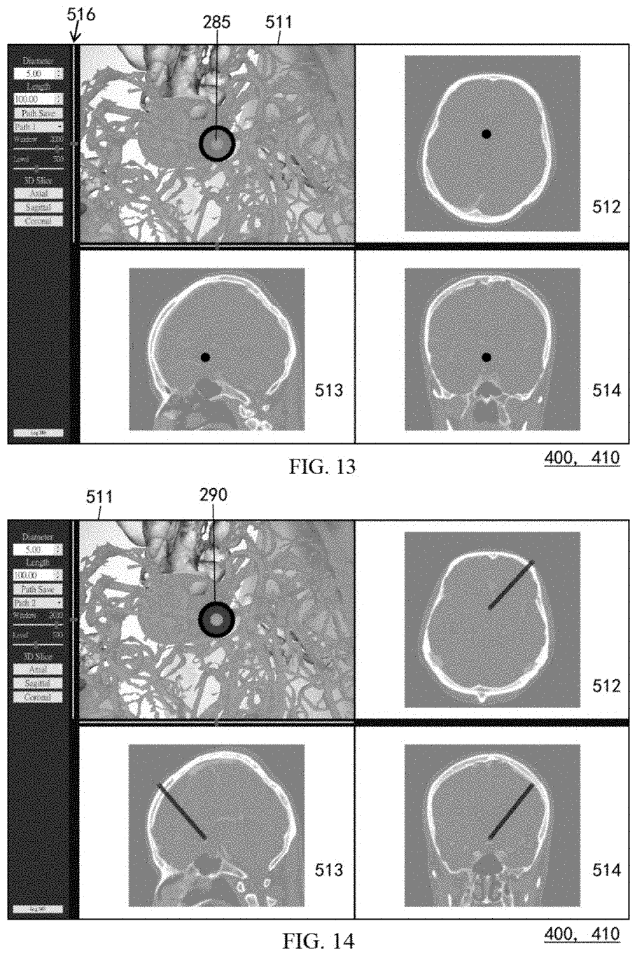

FIGS. 13 to 16 are schematic diagrams illustrating the actual operation process of preoperative surgical path planning of the aneurysm surgery applying the second embodiment of the three-dimensional surgical path simulator according to the present invention. The second embodiment is implemented based on the first embodiment and includes the first embodiment. In the second embodiment, the graphical user interface 410 of the three-dimensional surgical path simulator 400 according to the present invention includes four the three-dimensional medical model display windows 511, 512, 513, and 514 (or four viewable zone components for medical model). The aneurysm surgery is taken as an example to illustrate the implementation process of the three-dimensional surgical path simulator according to the present invention.

In this embodiment, preferably, the three-dimensional medical model display window 511 is the main operating window, a scrolling key 516 is configured on the edge of the three-dimensional medical model display window 511, and the integrated brain medical model 280 is loaded into the three-dimensional medical model display window 511 for the doctor to operate. Preferably, the three-dimensional medical model display windows 512, 513 and 514 are auxiliary windows for displaying the corresponding medical images (such as but not limited to X-ray profile images) of the integrated brain medical model 280 on the coronal, sagittal, and axial reference sections, respectively, to provide the doctor to interactively compare during planning of the surgical path and confirm the safety and feasibility of surgical path.

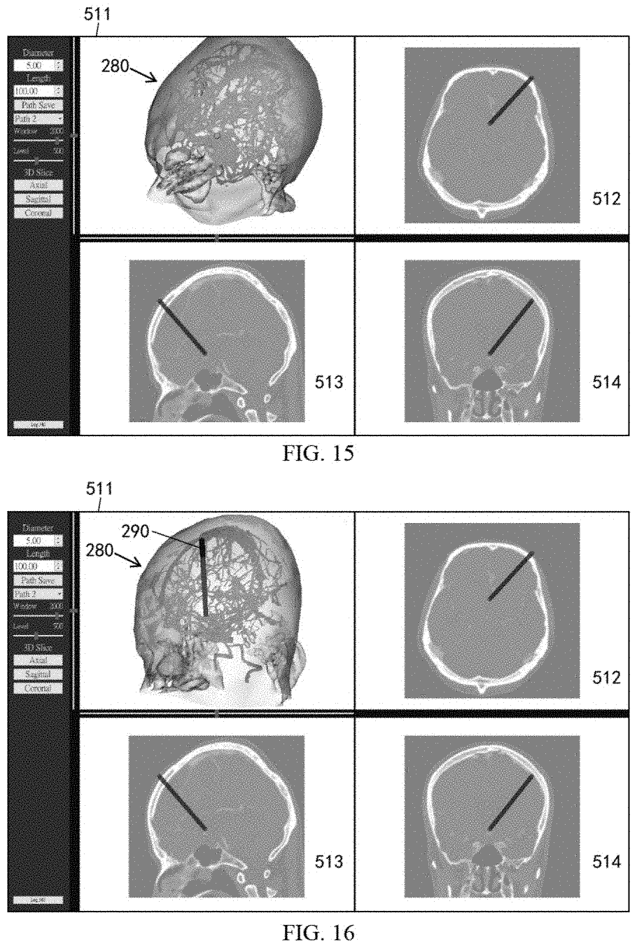

FIG. 13 is a schematic diagram illustrating the position of the aneurysm 285 in the integrated brain medical model 280. The system marks the aneurysm 285 with, for example but not limited to, black dots in the three-dimensional medical model display windows 512, 513 and 514. FIG. 14 shows the virtual three-dimensional object 290 just being inserted into the integrated brain medical model 280. The tubular virtual three-dimensional object 290 is displayed on the three-dimensional medical model display windows 512, 513 and 514. FIGS. 15 and 16 show that the doctor initially finds a surgical path and rotates the integrated brain medical model 280 together with the virtual three-dimensional object 290 to check the planned surgical path from different perspective.

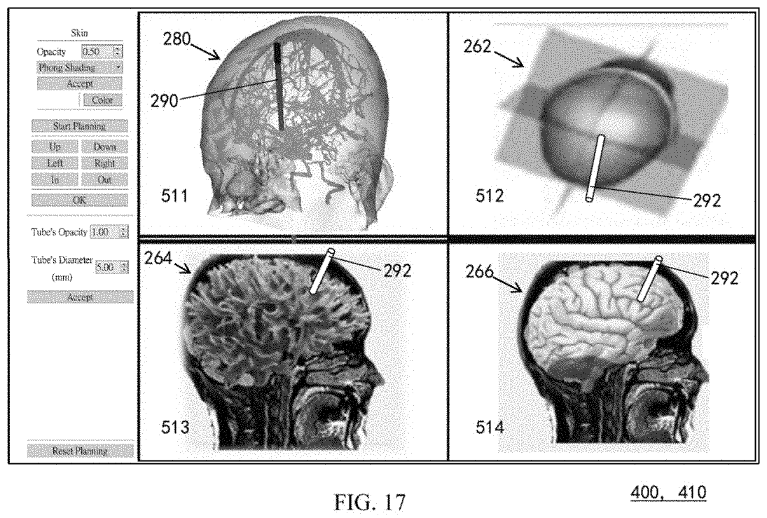

FIG. 17 is a schematic diagram illustrating the third embodiment of three-dimensional surgical path simulator according to the present invention. The third embodiment is implemented based on the first and second embodiments and includes the first and second embodiments. In this embodiment, the main operating window of the three-dimensional surgical path simulator 400 is the three-dimensional medical model display window 511. As mentioned above, the main operating window is provided to the doctor for planning of the surgical path. The other three auxiliary windows provide different functions, for example, the three-dimensional medical model display window 512 displays the three-dimensional head full digital model 262 and the virtual three-dimensional object 292, which are the simplified model of the integrated brain medical model 280 and the virtual three-dimensional object 290 displayed on the three-dimensional medical model display window 511. The three-dimensional head full digital model 262 and the virtual three-dimensional object 292 generate completely corresponding actions in response to the doctor's various operations on integrated brain medical model 280 and virtual three-dimensional object 290 in the three-dimensional medical model display window 511, to allow the doctor to compare during the surgical path planning process and confirm the safety and feasibility of surgical path.

The three-dimensional medical model display window 513 displays the three-dimensional brain neural medical model 264 and the virtual three-dimensional object 292 which generate completely corresponding actions in response to the doctor's various operations on the integrated brain medical model 280 and the virtual three-dimensional object 290 displayed on the three-dimensional medical model display window 511. The three-dimensional medical model display window 513 displays the three-dimensional brain sulcus (brain wrinkle) medical model 266 and the virtual three-dimensional object 292 which generate the partially-corresponding actions in response to the doctor's various operations on the integrated brain medical model 280 and the virtual three-dimensional object 290 displayed on the three-dimensional medical model display window 511.

In the fourth embodiment, the surgeon first completes the registration of the 3D models including the cerebrovascular model, the cranial nerve model, and the sulcus (brain wrinkle) model on the server, to complete the alignment of the skull model with the individual one or integrated one of the 3D models. After the registration is completed, the surgeon operates the surgical path planning computer-assisted processing device to load and display the 3D model on the three-dimensional surgical path simulator. The user first selects the target point and the entry point of the surgical path in the data set of the computer-readable three-dimensional visualization medical representation, and then the three-dimensional surgical path simulator generates the virtual three-dimensional object representing the surgical path and features thereof according to connection between the target point and the entry point, and the size and orientation of the path are displayed on the display windows simultaneously. The doctor can adjust the relative position of the virtual three-dimensional object with respect to the 3D model in any one of the four 3D windows, and the paths displayed on the other three 3D windows are also adjusted and displayed synchronously, so as to provide the doctor to interactively compare during planning of the surgical path and confirm the safety and feasibility of surgical path until the path desired by the surgeon is obtained.

In the fifth embodiment, each window can display one or more of three-dimensional image models of cerebrovascular model, brain wrinkles (sulcus), cranial nerves and lesions such as but not limited to aneurysms, pituitary tumors, neurofibromas, blood clots, cerebral edema, and these models can be aligned and merged to display in the window for planning of the surgical path. In each window, the displayed image model can be rotated, translated, zoomed in and zoomed out, and the tubular surgical path planned by the doctor can be integrally displayed.

Preferably, the planning of surgical path can be implemented by the following method. First, the doctor selects the target point of the surgical path at the location of the lesion displayed in the three-dimensional window. The surgical path simulator generates the virtual three-dimensional object representing for the surgical path and the feature thereof based on the target point and the doctor's visual line of sight toward the target point. The diameter of the cylindrical virtual three-dimensional object can be determined according to the surgical instrument used during the operation, and the virtual three-dimensional object is displayed on each of the 3D windows in the corresponding orientation simultaneously. The user can adjust the relative position and relative direction of the virtual three-dimensional object with respect to the data set of computer-readable three-dimensional visualization medical images, to simulate the surgical path. The images of the virtual three-dimensional object displayed on the other 3D windows are also adjusted and displayed simultaneously.

Preferably, the surgical path can be adjusted in the following method. The operation of adjusting the direction of the virtual three-dimensional object representing the surgical path is set to rotate around the selected target point. The rotation direction includes upward direction, downward direction, leftward direction or rightward direction. The virtual three-dimensional object can also be adjusted forwardly (toward the target point) or backwardly (away from the target point) to avoid the three-dimensional tissue image model of cerebrovascular model or cranial nerves, and the best surgical path is toward the sulcus (brain wrinkle). The system can also be in cooperation with the AR glasses to perform the above-mentioned path planning in real 3D environment.

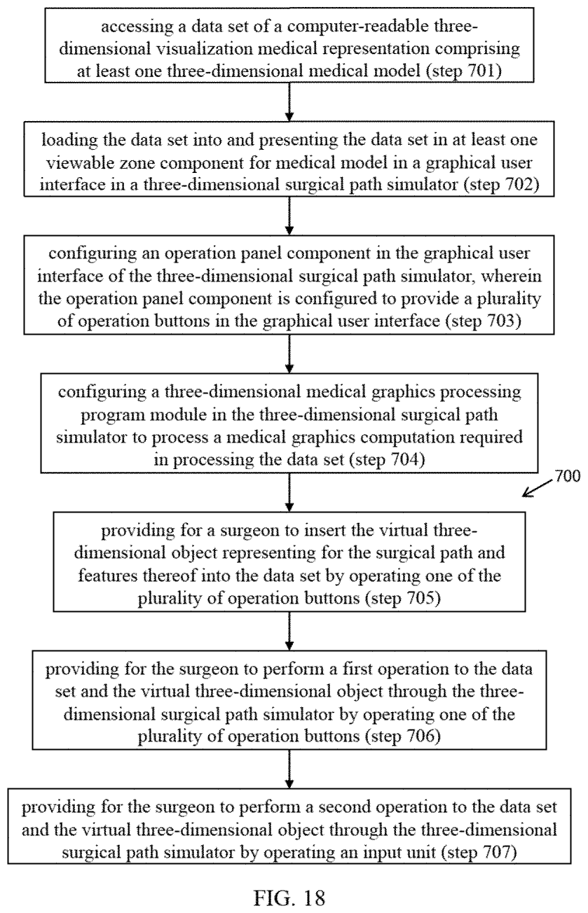

FIG. 18 is a flow chart illustrating the computer-implemented method for a computer-assisted planning of a surgical path according to the present invention. To sum up, the computer-implemented method 700 for a computer-assisted planning of a surgical path in accordance with the present invention preferably includes the following steps: accessing a data set of a computer-readable three-dimensional visualization medical representation comprising at least one three-dimensional medical model (step 701); loading the data set into and presenting the data set in at least one viewable zone component for medical model in a graphical user interface in a three-dimensional surgical path simulator (step 702); configuring an operation panel component in the graphical user interface of the three-dimensional surgical path simulator, wherein the operation panel component is configured to provide a plurality of operation buttons in the graphical user interface (step 703); configuring a three-dimensional medical graphics processing program module in the three-dimensional surgical path simulator to process a medical graphics computation required in processing the data set (step 704); providing for a surgeon to insert the virtual three-dimensional object representing for the surgical path and features thereof into the data set by operating one of the plurality of operation buttons (step 705); providing for the surgeon to perform a first operation to the data set and the virtual three-dimensional object through the three-dimensional surgical path simulator by operating one of the plurality of operation buttons (step 706); and providing for the surgeon to perform a second operation to the data set and the virtual three-dimensional object through the three-dimensional surgical path simulator by operating an input unit (step 707).

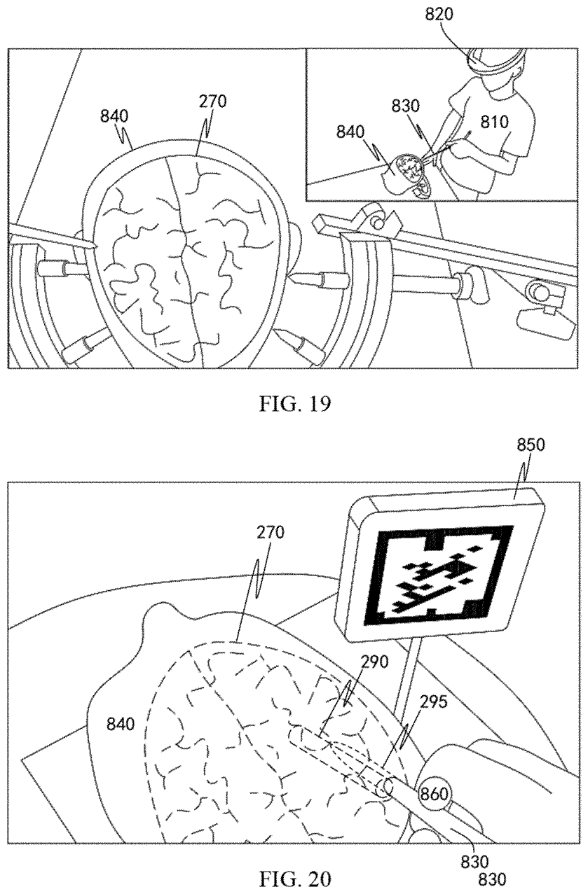

FIG. 19 is a schematic diagram illustrating the scenario applying the surgical path planned by the present invention in an augmented reality surgical navigation system, and a schematic diagram of doctor's AR view in which the surgical path planned by the present invention, is overlapped with the reality; FIG. 20 is a schematic diagram illustrating that the surgical path planned by the present invention is projected and overlapped with the reality through AR surgical navigation system. The surgical path planned by the present invention can be further used in the augmented reality (AR) surgical navigation system as the surgical navigation path, as shown in FIG. 20, and the surgeon 810 wears AR glasses 820 and prepares to perform the resection for aneurysm in patient's head 840 by using the surgical instrument 830.

FIGS. 19 and 20 shows the scenario in which the surgeon 810 watches the reality and the augmented reality overlapped with the reality through the AR glasses 820. The AR glasses 820 projects the three-dimensional digital medical model (preferably, the integrated medical model 270 for a target lesion) of the target lesion model on the patient's head 840 by sensing the position of the target marker 850 to correctly sense the position of the patient's head 840. The AR surgical navigation system correctly senses the position of the surgical instrument by tracking the position marker 860 disposed on the surgical instrument 830. For detailed technical content of the augmented reality surgical navigation system including cooperation between the target marker, the position marker and the surgical navigation system, please refer to the applicant's Taiwan Invention Patent Application No. 108131367, titled “Digital Image Reality Alignment Kit and Method Applied to Mixed Reality System for Surgical Navigation”, and the Taiwan Invention Patent Application No. 108131391, titled “Digital Image Reality Alignment Kit and Method Applied to Mixed Reality System for Surgical Navigation”, which are incorporated herein by reference.

During the surgery, the AR glasses 820 continuously projects the well-positioned and well-aligned virtual three-dimensional object 290, which is preplanned, represents for the surgical path and is preferably displayed by blue color, on the patient head 840 as the surgical navigation path, which corresponds to the integrated medical model 270 for a target lesion correctly, to facilitate the surgeon 810 to operate the surgical instrument 830 to perform surgery. After the surgical instrument 830 enters the patient's cranium, the AR glasses 820 projects the virtual surgical instrument object 295, which represents for the surgical instrument 830 and is preferably displayed by green color, on the patient's head 840, and the integrated medical model 270 for a target lesion also corresponds to the patient's head 840 correctly. At this time, through the AR glasses 820, the surgeon 810 can check whether the virtual surgical instrument object 295 deviates from the virtual three-dimensional object 290 serving as the surgical navigation path, and when the offset distance between the virtual surgical instrument object 295 and the virtual three-dimensional object 290 exceeds a preset threshold, the virtual three-dimensional object 290 serving as the surgical navigation path is changed to be displayed in red color, and the AR glasses 820 simultaneously makes a warning sound, to remind the surgeon 810 to immediately correct the path of the surgical instrument 830.

The 3D vision perspective surgical path planning platform according to the present invention construct the 3D digital skull model based on patient's head medical images, such as but not limited to: CT images, CTA images, MR images, MRA images, FA images, DTI images or DWI images, etc. and the 3D digital skull model includes the skin model, the skull model, the sulcus model, the lesion model, the aneurysm model, the cerebral blood clot model, the cerebral edema model, the cerebrovascular model, the cranial nerve model and so on. The constructed 3D digital skull model is not just an image model but is able to clearly present the spatial distributions of brain sulcus, blood vessels, nerves and lesion in the skull, and accurately mark the position of the lesion. Therefore, the doctor can operate the 3D digital model through one of the plurality of operation buttons on the GUI control panel, to easily plan the best and safest surgical path in a very relaxed, intuitive and intuitive way. Furthermore, the platform can be further in cooperation with AR, VR, and 3D display technology, to plan a more real 3D surgical path.

The present invention has the following advantages: (1) the number of X-ray image images required for surgery can be greatly decreased; (2) the patient's radiation exposure is greatly reduced; (3) the success rate of surgery can be increased and the risk of surgery can be decreased; (4) a truly safe and accurate surgical path can be planned and simulated according to the present invention; (5) the patient can obtain an easy-to-understand symptom descriptions, so as to promote the communication between doctors and the patient; (6) all important tissues of the target lesion including arteries, veins, nerve plexus and tissues can be viewed; (7) the present invention can be in cooperation with the AR surgical navigation system for better performance; (8) the present invention is easy to in cooperation with VR-based medical technology; (9) The doctor can easily determine whether the surgical path is basically feasible and safe; (10) the present invention can provide the Intuitive operation in the three-dimensional environment.

There are further embodiments provided as follows.

Embodiment 1: A computer-implemented method for a computer-assisted planning of a surgical path, the method includes accessing a data set of a computer-readable three-dimensional visualization medical representation including at least one three-dimensional medical model; loading the data set into and presenting the data set in a three-dimensional surgical path simulator; inserting a virtual three-dimensional object representing for the surgical path and features thereof into the data set; and adjusting a relative position of the virtual three-dimensional object with respect to the at least one three-dimensional medical model.

Embodiment 2: The computer-implemented method as described in Embodiment 1, further includes configuring an operation panel component in a graphical user interface of the three-dimensional surgical path simulator, wherein the operation panel component is configured to provide a plurality of operation buttons in the graphical user interface; configuring at least one viewable zone component for medical model in the graphical user interface to display the at least one three-dimensional medical model included in the data set; configuring a three-dimensional medical graphics processing program module in the three-dimensional surgical path simulator to process a medical graphics computation required in processing the data set; inserting the virtual three-dimensional object representing for the surgical path and features thereof into the data set by operating one of the plurality of operation buttons; performing a first operation to the data set and the virtual three-dimensional object through the three-dimensional surgical path simulator by operating one of the plurality of operation buttons; and performing a second operation to the data set and the virtual three-dimensional object through the three-dimensional surgical path simulator by operating an input unit.

Embodiment 3: The computer-implemented method as described in Embodiment 2, the first operation is selected from one of an operation to adjust a degree of opacity, an operation to enable a Phong shading, an operation to enable a three-dimensional rendering, an operation to move upward, an operation to move downward, an operation to move leftward, an operation to move rightward, an operation to zoom in, an operation to zoom out, an operation to magnify, an operation to shrink, an operation to enable resizing, an operation to enable scaling, an operation to insert an object, an operation to set dimensions, an operation to reset, and a combination thereof.

Embodiment 4: The computer-implemented method as described in Embodiment 2, the second operation is selected from one of an operation to zoom in, an operation to zoom out, an operation to magnify, an operation to shrink, an operation to enable resizing, an operation to enable scaling, an operation to enable translation, an operation to enable rotation, an operation to adjust a viewing angle, an operation to edit, an operation to modify, an operation to disable layer, an operation to enable layer, and a combination thereof.

Embodiment 5: The computer-implemented method as described in Embodiment 1, further includes pre-performing a three-dimensional medical graphics modelling algorithm to construct the at least one three-dimensional medical model by integrating a plurality of basic medical images; and storing the at least one three-dimensional medical model in a non-volatile memory unit or a medical data set server in a remote end.

Embodiment 6: The computer-implemented method as described in Embodiment 5, the three-dimensional medical graphics modelling algorithm is selected from one of a noise removal process, an image preprocessing, an anatomy based feature recognition, an anatomy based feature enhancement, an image aligning process for three-dimensional medical images, an image stitching process for three-dimensional medical images, an image interpolation process for three-dimensional medical images, an image extrapolation process for three-dimensional medical images, and a combination thereof.

Embodiment 7: The computer-implemented method as described in Embodiment 5, the plurality of basic medical images are selected from one of an X-ray image, a computerized tomography (CT) image, a digital subtraction angiography (DSA) image, a maximum intensity projection (MIP) image, a magnetic resonance imaging (MRI) image, a magnetic resonance angiography (MRA) image, a functional anisotropy (FA) image, a diffusion tensor imaging (DTI) image, a diffusion-weighted imaging (DWI) image, and a combination thereof.

Embodiment 8: The computer-implemented method as described in Embodiment 1, the at least one three-dimensional medical model is selected from an integrated medical model, a target lesion medical model, a skin medical model, a blood medical model, a neural medical model, and a skeleton medical model.

Embodiment 9: A non-transitory computer-readable medium stores a program causing a computer to execute a computer-implemented method for a computer-assisted planning of a surgical path, and the method includes in response to a user's operation, accessing a data set of a computer-readable three-dimensional visualization medical representation including at least one three-dimensional medical model; loading the data set into and presenting the data set in a three-dimensional surgical path simulator; inserting a virtual three-dimensional object representing for the surgical path and features thereof into the data set; and adjusting a relative position of the virtual three-dimensional object with respect to the at least one three-dimensional medical model.

Embodiment 10: A computerized processing device for a computer-assisted planning of a surgical path, the device includes a processing unit configured to load and execute a three-dimensional surgical path simulator, in which the three-dimensional surgical path simulator accesses a data set of a computer-readable three-dimensional visualization medical representation including at least one three-dimensional medical model in response to a user's operation; a display unit configured to present the three-dimensional surgical path simulator and the data set; and an input unit configured to provide for a user to operate to insert a virtual three-dimensional object representing for the surgical path and features thereof into the data set and adjust a relative position of the virtual three-dimensional object with respect to the at least one three-dimensional medical model.

Embodiment 11: The computerized processing device as described in Embodiment 10, further includes a non-volatile memory configured to store the data set for the three-dimensional surgical path simulator to access; and a medical data set server in a remote end configured to separate from but communicatively connect with the processing unit and configured to store the data set for the three-dimensional surgical path simulator to access.

Embodiment 12: The computerized processing device as described in Embodiment 10, the input unit is selected from a mouse and a keyboard.

While the disclosure has been described in terms of what are presently considered to be the most practical and preferred embodiments, it is to be understood that the disclosure need not be limited to the disclosed embodiments. On the contrary, it is intended to cover various modifications and similar arrangements included within the spirit and scope of the appended claims, which are to be accorded with the broadest interpretation so as to encompass all such modifications and similar structures. Therefore, the above description and illustration should not be taken as limiting the scope of the present disclosure which is defined by the appended claims.

1. A computer-implemented method for a computer-assisted planning of a surgical path, the method comprising:
loading and displaying a data set of a computer-readable three-dimensional visualization medical representation comprising at least one three-dimensional medical model and a lesion into a three-dimensional surgical path simulator;
providing for a user to operate the three-dimensional surgical path simulator to select a point from the lesion as a target point, to generate a virtual three-dimensional object representing for the surgical path and features thereof along a line of sight of the user started from the target point; and
adjusting at least one geometrical parameter and a relative position and direction of the virtual three-dimensional object with respect to the at least one three-dimensional medical model by operating the three-dimensional surgical path simulator to obtain a safe surgical path.

2. The computer-implemented method as claimed in claim 1, further comprising one of following steps:
accessing the data set of the computer-readable three-dimensional visualization medical representation;
configuring an operation panel component in a graphical user interface of the three-dimensional surgical path simulator, wherein the operation panel component is configured to provide a plurality of operation buttons in the graphical user interface;
configuring at least one viewable zone component for medical model in the graphical user interface to display the at least one three-dimensional medical model comprised in the data set;
configuring a three-dimensional medical graphics processing program module in the three-dimensional surgical path simulator to process a medical graphics computation required in processing the data set;
generating the virtual three-dimensional object representing for the surgical path and features thereof into the data set by operating one of the plurality of operation buttons;
performing a first operation to the data set and the virtual three-dimensional object through the three-dimensional surgical path simulator by operating one of the plurality of operation buttons; and
performing a second operation to the data set and the virtual three-dimensional object through the three-dimensional surgical path simulator by operating an input unit.

3. The computer-implemented method as claimed in claim 2, wherein the first operation is selected from one of an operation to adjust a degree of opacity, an operation to enable a Phong shading, an operation to enable a three-dimensional rendering, an operation to rotate upward, an operation to rotate downward, an operation to rotate leftward, an operation to rotate rightward, an operation to zoom in, an operation to zoom out, an operation to magnify, an operation to shrink, an operation to enable resizing, an operation to enable scaling, an operation to insert an object, an operation to set dimensions, an operation to reset, and a combination thereof.

4. The computer-implemented method as claimed in claim 2, wherein the second operation is selected from one of an operation to zoom in, an operation to zoom out, an operation to magnify, an operation to shrink, an operation to enable resizing, an operation to enable scaling, an operation to enable translation, an operation to enable rotation, an operation to adjust a viewing angle, an operation to edit, an operation to modify, an operation to disable layer, an operation to enable layer, and a combination thereof.

5. The computer-implemented method as claimed in claim 1, further comprising:
pre-performing a three-dimensional medical graphics modelling algorithm to construct the at least one three-dimensional medical model by integrating a plurality of basic medical images; and
storing the at least one three-dimensional medical model in a non-volatile memory unit or a medical data set server in a remote end.

6. The computer-implemented method as claimed in claim 5, wherein the three-dimensional medical graphics modelling algorithm is selected from one of a noise removal process, an image preprocessing, an anatomy based feature recognition, an anatomy based feature enhancement, an image aligning process for three-dimensional medical images, an image stitching process for three-dimensional medical images, an image interpolation process for three-dimensional medical images, an image extrapolation process for three-dimensional medical images, an image reconstruction process for three-dimensional medical images, and a combination thereof.

7. The computer-implemented method as claimed in claim 5, wherein the plurality of basic medical images are selected from one of an X-ray image, a computerized tomography (CT) image, a digital subtraction angiography (DSA) image, a maximum intensity projection (MIP) image, a magnetic resonance imaging (MRI) image, a magnetic resonance angiography (MRA) image, a functional anisotropy (FA) image, a diffusion tensor imaging (DTI) image, a diffusion-weighted imaging (DWI) image, and a combination thereof.

8. The computer-implemented method as claimed in claim 1, wherein the at least one three-dimensional medical model is selected from an integrated medical model, a lesion medical model, a skin medical model, a vessel medical model, a nerve medical model, and a skeleton medical model, and the at least one geometrical parameter is selected from one of a length, a shape, a diameter, a radius, and a combination thereof.

9. A non-transitory computer-readable medium storing a program causing a computer to execute a computer-implemented method for a computer-assisted planning of a surgical path, the method comprising:
in response to a user's operation, loading and displaying a data set of a computer-readable three-dimensional visualization medical representation comprising at least one three-dimensional medical model and a lesion into a three-dimensional surgical path simulator;
providing for a user to operate the three-dimensional surgical path simulator to select a point from the lesion as a target point, to generate a virtual three-dimensional object representing for the surgical path and features thereof along a line of sight of the user started from the target point; and
adjusting at least one geometrical parameter and a relative position and direction of the virtual three-dimensional object with respect to the at least one three-dimensional medical model by operating the three-dimensional surgical path simulator to obtain a safe surgical path.

10. A computerized processing device for a computer-assisted planning of a surgical path, the device comprising:
a processing unit configured to load and execute a three-dimensional surgical path simulator, in which the three-dimensional surgical path simulator loads and displays a data set of a computer-readable three-dimensional visualization medical representation comprising at least one three-dimensional medical model and a lesion in response to a user's operation;
a display unit configured to present the three-dimensional surgical path simulator and the data set; and
an input unit configured to provide for a user to operate the three-dimensional surgical path simulator to select a point from the lesion as a target point, to generate a virtual three-dimensional object representing for the surgical path and features thereof along a line of sight of the user started from the target point to generate a virtual three-dimensional object representing for the surgical path and features thereof into the data set and adjust at least one geometrical parameter and a relative position and direction of the virtual three-dimensional object with respect to the at least one three-dimensional medical model to obtain a safe surgical path.

11. The computerized processing device as claimed in claim 10, further comprising:
a non-volatile memory configured to store the data set for the three-dimensional surgical path simulator to access; and
a medical data set server in a remote end configured to separate from but communicatively connect with the processing unit and configured to store the data set for the three-dimensional surgical path simulator to access.

12. The computerized processing device as claimed in claim 10, wherein the input unit is selected from a mouse, a stylus pen, and a keyboard.
